(19) United States
(12) Patent Application Publication
(10) Pub. No.: US 2005/0004527 A1
(43) Pub. Date: Jan. 6, 2005

(54) Apparatus and method for containing and dispensing medical grade sterile viscous substances

(76) Inventor: Albert Prescott, Westford, MA (US)

Correspondence Address: Brian M. Dingman, Esq.;Mirick, O'Connell, DeMallie & Lougee, LLP, 1700 West Park Drive, Westborough, MA, 01581-3941, US

(21) Appl. No.: 10/844,263

(22) Filed: May 12, 2004

Related U.S. Application Data
(60) Provisional application No. 60/470,014, filed on May 13, 2003.

(51) Int. Cl. A61M 31/00

(52) U.S. Cl. 604/181

(57) ABSTRACT

An apparatus containing and dispensing medical grade sterile viscous substances comprised of a storage container resembling a syringe of a volume 1 to 60 or greater milliliters, a plunger similar to that used in syringes, and a needless septum. The apparatus is designed to meter out varying volumes of the material, while maintaining sterility of the contents in the storage container. The method involves connecting a receiving container (typically a syringe without a needle) to the container through the needless septum, and pulling on the plunger in the receiving syringe and/or pushing on the plunger in the container, to transfer a desired amount of the material from the container, through the septum, and into the receiving container.

### RELATED APPLICATIONS

This application claims benefit of U.S. Provisional Application No. 60/470,014, filed May 13, 2003.

### FIELD OF THE INVENTION

This invention relates to the packaging, storing, supplying and dispensing of medical grade sterile viscous substances.

### BACKGROUND OF THE INVENTION

Sterile viscous substances are being used increasingly in the medical field. These substances have found application in ophthalmic surgeries, synovial fluid replacement in the knee and other joints, incontinence treatment and a variety of other applications. These substances can include hyaluronic acid, carboxyl-methyl-cellulose, poly-glutamic acid, collagen, gelatin, and other similar materials. Unlike non-viscous materials, such viscous substances do not flow of their own accord. Therefore, special consideration must be taken when dealing with them.

To date, medical products made of sterile viscous substances cannot be sold in the typical way other sterile liquids are sold. Sterile liquids are typically sold in a glass or plastic jar with a septum. The septum is made of rubber. To dispense the contents into a syringe, the septum is sterilized by swabbing it with alcohol. The septum is then penetrated with a sterile needle attached to a sterile syringe, the jar is inverted, and the liquid is removed into the syringe via suction of the syringe.

Viscous substances cannot be sold packaged like this, because they do not flow to the bottom of the jar when inverted, and the maximum suction that a syringe can generate (14.7 psia at full vacuum) is often not enough to “pull” the viscous material through the small diameter of the needle into the syringe. Even if a large enough needle is used, viscous substances don't flow gently into needles, rather they cling to the sides of the jar and form a hole in the middle, a phenomenon sometimes referred to as “rat-holing”. This introduces air into the syringe, which cannot be removed, and leaves large amounts of material in the jar, both of which are unacceptable results.

The most typical method for addressing this issue is to sell viscous substances “pre-filled” into a syringe, at the dose most commonly required for administration to the patient. In the event that more than one dose size is required, the company selling the material must choose to pre-fill this other amount. For instance, some opthalmics such as the product Amvisc®, are sold pre-filled at 0.5 ml and 0.8 ml, depending on the preference of the surgeons using the product. Other materials, such as Synvisc® and Hyalgan®, are sold only as 2 ml pre-fills because that is the amount used to treat osteoathritis of the knee. When treating other joints that require more or less material, the user must either select multiple pre-filled syringes, and thus carry the cost of two syringes, or use less than the pre-filled amount, and thus carry the cost of the discarded material.

In addition to these previously mentioned fields and products, a whole new field of extended release drug delivery has yet to be realized. In this field, drugs are formulated in the presence of a polymer matrix. These matrixes are typically viscous in nature. In order to properly treat the patient, dose modifications must be made taking into account the weight of the patient, thus maintaining a recommended mg-drug per kg-body weight (mg/kg). This fact has thus far made viscous polymer-drug delivery impractical and costly, since pre-filled syringes will not suffice.

### SUMMARY OF THE INVENTION

Given the state of the art, there is a need for a package that allows a doctor or other medical professional to selectively withdraw a chosen amount of sterile viscous material from a container, without compromising the sterility of the remaining material in the container. The package should also have the ability to dispense all of the material, without introducing air bubbles.

This invention features a storage container resembling a syringe of a volume 1 to 60 (or greater) milliliters, a plunger similar to that used in syringes, and a needless septum similar to the NAC-PRN made by Porex. The inventive apparatus is designed to meter out varying volumes of the material with minimal air entrainment, while maintaining sterility of the contents in the main container.

### BRIEF DESCRIPTION OF THE DRAWINGS

FIG. 1 is a photograph of an embodiment of the inventive apparatus described below, which uses an adapter between the container and the septum.

FIG. 2 is a schematic cross-sectional drawing of an embodiment in which the septum is directly connected to the container.

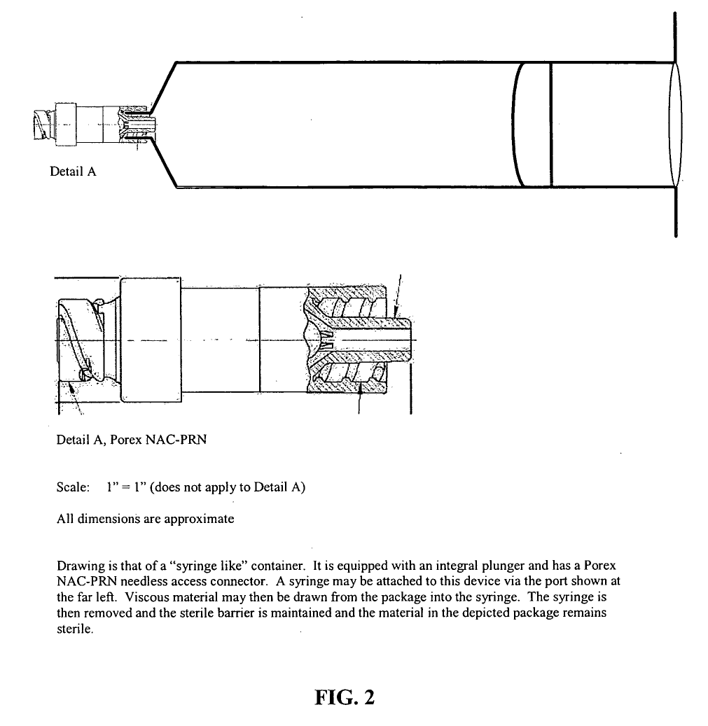

### DETAILED DESCRIPTION OF THE PREFERRED EMBODIMENTS

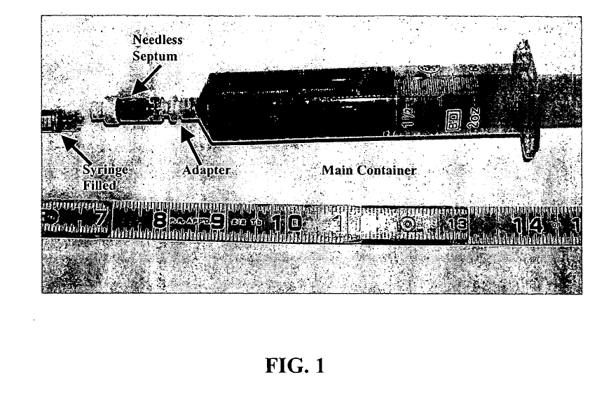

The following is a description of the preferred embodiment of the invention. The preferred container is 60 milliliters, though a person skilled in the art will recognize that the container is not restricted to this volume. The container may resemble, or be, a syringe 10, complete with a plunger 12. As material is withdrawn from the container, the plunger is operated, to assure that the material does not cling to the sidewalls of the container, no rat-hole is formed, and the material empties completely from the container. A needless septum 14, preferably an “NAC-PRN” made by Porex, is used to maintain the sterile barrier between the environment and the contents of the container. The syringe being filled (shown in FIG. 1) does not have a needle. Thus, there is a passageway that is sufficiently large such that full vacuum is enough to pull the viscous material from the container into the syringe, while eliminating contamination with air bubbles.

The needless septum may be attached to the container in a variety of manners including solvent welding, ultrasonic welding, or one-piece molding. In the event that these methods are not desired, an adapter may also be used. The embodiment shown in FIG. 1 uses a female lure to female lure adapter, one example of such being an “FLL-FLL” supplied by Harvard Apparatus, between the container and the needless septum. This is a convenience which allows a commercially-available septum to be coupled to a commercially-available container syringe.

1. An apparatus for containing and dispensing medical grade sterile viscous substances, comprising:
a storage container having an output;
a plunger in the container; and
a needless septum connected to the container output.

2. The apparatus of claim 1, wherein the container is greater than 1 milliliter in volume.

3. The apparatus of claim 1, wherein the needless septum is an “NAC-PRN” by Porex.

4. The apparatus of claim 1, wherein the septum is attached to the container via solvent welding.

5. The apparatus of claim 1, wherein the septum is attached to the container via ultrasonic welding.

6. The apparatus of claim 1, wherein the septum is attached to the container via molding.

7. The apparatus of claim 1, wherein the septum is attached to the container via an adapter.

8. The apparatus of claim 7, wherein the adapter is a female lure to female lure adapter.

9. The apparatus of claim 8, wherein the adapter is an “FLL-FLL” by Harvard Apparatus.
